(19) United States
(12) Patent Application Publication
(10) Pub. No.: US 2013/0196920 A1
(43) Pub. Date: Aug. 1, 2013

(54) PROCESS FOR PRODUCTION OF BIVALIRUDIN

(71) Applicant: TEVA Pharmaceuticals USA, Inc., North Wales, PA (US)

(72) Inventors: Avi TOVI, Zurit (IL); Chaim EIDELMAN, Oshrat (IL); Shimon SHUSHAN, Karmiel (IL); Alon HAGI, Kibbutz Yehiam (IL); Alexander IVCHENKO, Keryat Ata (IL); Gabriel-Marcus BUTILCA, Karmiel (IL); Leah BAR-OZ, Kiryat Bialik (IL); Tehila GADI, Kfar Vradim (IL); Gil ZAOVI, Keryat Bialik (IL)

(73) Assignee: TEVA Pharmaceuticals USA, Inc., North Wales, PA (US)

(21) Appl. No.: 13/850,221

(22) Filed: Mar. 25, 2013

Related U.S. Application Data
(63) Continuation of application No. 12/536,281, filed on Aug. 5, 2009.
Application No. 12/536,281 is a division of application No. 11/521,968, filed on Sep. 14, 2006, now abandoned.
(60) Provisional application No. 60/717,442, filed on Sep. 14, 2005.

(51) Int. Cl. C07K 14/815; A61K 38/58

(52) U.S. Cl. CPC C07K 14/815; A61K 38/58
USPC 514/14.7; 530/326

(57) ABSTRACT

The invention relates to methods for the preparation of high purity Bivalirudin. The polypeptide is prepared in a high purity of at least 98.5% (by HPLC), wherein the total impurities amount to less than 1.5%, comprising not more than 0.5% [Asp⁹-Bivalirudin] and each is impurity less than 1.0%, and preferably having a purity of at least about 99.0% by HPLC, wherein the total impurities amount to less than 1.0%, comprising not more than 0.5% [Asp⁹-Bivalirudin] and each impurity is less than 0.5%.

### CROSS REFERENCE TO RELATED APPLICATIONS

This application is a continuation of U.S. patent application Ser. No. 12/536,281, filed Aug. 5, 2009, which is a division of U.S. patent application Ser. No. 11/521,968, filed Sep. 14, 2006, abandoned, which claims the benefit of the following U.S. Provisional Patent Application No.: 60/717,442, filed Sep. 14, 2005. The content of these applications is incorporated herein by reference.

### FIELD OF THE INVENTION

The present invention is related to an improved process for the preparation of Bivalirudin. Furthermore it encompasses highly pure Bivalirudin.

### BACKGROUND OF THE INVENTION

Proteolytic processing by thrombin is pivotal in the control of blood clotting and indicated as an anticoagulant in patients with unstable angina undergoing percutaneous transluminal coronary angioplasty (PTCA) or as an anticoagulant in patients undergoing percutaneous coronary intervention. Hirudin, a potential clinical thrombin peptide inhibitor from the blood sucking leech, Hirudo medicinalis, consists of 65 amino acids, while shorter peptide segment amino acids have proven effective in treatment of thrombosis, a life threatening condition.

U.S. patent application Ser. No. 5,196,404, discloses, amongst others, one of these shorter peptides, a potent thrombin inhibitor such as Bivalirudin, also known as Hirulog-8, having the following chemical name: D-phenylal-anyl-L-prolyl-L-arginyl-L-prolyl-glycyl-glycyl-glycyl-glycyl-L-asparagyl-glycyl-L-aspartyl-L-phenylalanyl-L-glutamyl-L-glutamyl-L-isoleucyl-L-prolyl-L-glutamyl-L-glutamyl-L-tyrosyl-L-leucine trifluoroacetate (salt) hydrate (SEQ ID No:7) and is made up of the following amino acid sequence: H-D-Phe-Pro-Arg-Pro-Gly-Gly-Gly-Gly-Asn-Gly-Asp-Phe-Glu-Glu-Ile-Pro-Glu-Glu-Tyr-Leu-OH, (SEQ ID No:1).

Other common names include: hirulog-8, BG-8967, Efludan, Angiomax® and Hirulog®.

PCT Patent Application WO98/50563 apparently describes a method for production of various peptides, including Hirulog by a recombinant technology. The method comprises expressing the peptide as part of a fusion protein (FP), followed by the release of the peptide from the FP by an acyl-acceptor, such as a sulphur containing reductant.

Okayama et al. (1996, Chem Pharm. Bull. 44:1344-1350) and Steinmetzer et al. (1999, Eur. J. Biochem. 265:598-605) devise solid phase synthesis of different Hirulogs on Wang resin. The Wang resin requires cleavage of the peptide from the resin with concentrated trifluoroacetic acid. In similar solid phase synthesis approach for the preparation of Bivalirudin, PCT Patent Application WO91/02750 apparently discloses a sequential approach of adding single BOC-protected amino acids on a solid phase of BOC-L-Leucine-O-divinylbenzene resin, simultaneous deprotecting and uncoupling using HF/p-cresol/ethylmethyl sulfate; lyophilising and purifying the crude Hirulog-8. Cleavage from the resin in both cases described require aggressive acidic conditions which is likely to cause concomitant global deprotection of peptide and incur undesirable side reaction with amino acid residues, despite the use of scavenging reagents, thus affecting product purity.

Purity of the active compound is an extremely important parameter specifically for products used as APIs (active pharmaceutical ingredients). Various grades of purity of the same product are possible at the end of the production process. In general, the purity of the product depends on the chemistry and various process related parameters of the production process. In the case of peptide products the situation is even more complicated as peptides are complex and sensitive molecules. They are produced by multi-step processes applying an extensive variety of starting materials and are potentially contaminated due to the many possible side reactions, which are part of peptide chemistry.

Thus, it is the object of the present invention to devise other and especially improved methods of synthesizing the respective Bivalirudin peptides that lacks the disadvantages of the prior art.

Thus the production of a high purity peptide product is a highly desired but difficult to achieve goal. In fact, only specially designed processes developed to produce such high purity products can be used to achieve this target. The present invention provides such process of preparing the Bivalirudin peptide in a high purity.

### SUMMARY OF THE INVENTION

The present invention encompasses improved methods of synthesizing the Bivalirudin peptides that lacks the disadvantages of the prior art. The method of production can be based on a solid phase synthesis or a combination of solid phase and solution synthesis (hybrid approach). The synthesis of the peptide chain can be performed sequentially or by coupling of two or more short fragments to form a final sequence of a Bivalirudin molecule. These fragments can be prepared in solution or on solid support in protected, partially protected, or unprotected form. Coupling between fragments can be performed through activation of the carboxyl group of one peptide fragment (C-terminus) to another fragment (N-terminus) by a suitable coupling reagent or other suitable method such as coupling through an active ester. After completion of the synthesis, side chain protecting groups are removed and the peptide is purified by a suitable method, such as preparative HPLC, to a high degree of purity.

In one embodiment, there is provided a process for the preparation of Bivalirudin comprising (a) preparing a Bivalirudin peptide sequence on a hyper acid-labile resin, wherein the peptide contains suitably protected amino acids; (b) treating the Bivalirudin peptide coupled to resin with an acid solution to obtain an unprotected or semi-protected crude peptide free of the resin; (c) in the case of semi-protected crude peptide, removing any remaining protecting groups; and (d) recovering the crude Bivalirudin peptide. Preferably, the crude Bivalirudin peptide is then purified.

In a particularly preferred embodiment of the present inventions, the suitably protected bivalirudin peptide sequence contain a-amino residues protected by Fmoc while other functional residues of the amino acids are protected with suitable acid stable protecting groups.

In another embodiment, the process for the preparation of Bivalirudin comprises:

(a) providing a N-terminus protected peptide fragment A of Bivalirudin, preferably [Xα-D-Phe-Pro-Arg(X)-Pro-Gly-Gly-Gly-Gly-Asn(X)-Gly-OH] (SEQ ID No: 2), wherein Xα is a suitable a-amino protecting group, preferably BOC or FMOC, and X is a suitable protecting group, preferably Pbf for Arg and tBu or Trt for other residues, which fragment A is prepared on a hyper acid-labile resin and subsequently detached in protected form by treatment under mild acidic conditions, and is optionally isolated;

(b) providing a protected fragment B of Bivalirudin, preferably [FMOC-Asp(X)-Phe-Glu(X)-Glu(X)-Ile-Pro-Glu(X)-Glu(X)-Tyr(X)—OH] (SEQ ID No:3)—OR FMOC-FRAGMENT B, wherein X is a suitable protecting group, preferably tBu or Trt, which fragment B is prepared on a hyper acid-labile resin and subsequently detached in protected form by treatment under mild acidic conditions, and is optionally isolated;

(c) coupling of the fragment B with Leu-OtBu to form an elongated fragment B;

(d) deprotecting of the a-amino protecting group from the elongated fragment B;

(e) coupling of fragment A with the previously obtained elongated fragment B of step (d);

(f) deprotecting all remaining protecting groups from the peptide with a treatment in strong acidic solution.

Optionally the crude Bivalirudin is then isolated and purified to obtain Bivalirudin of high purity in high yield.

In another embodiment there is provided highly pure Bivalirudin having a purity of at least about 98.5%, preferably a purity of at least about 99.0%.

In another embodiment there is provided a pharmaceutical composition comprising highly pure Bivalirudin having a purity of at least about 98.5% and at least one pharmaceutical acceptable excipient.

In another embodiment there is provided a method of preparing a pharmaceutical composition comprising Bivalirudin having a purity of at least 98.5% comprising preparing Bivalirudin, either in fragments or in its entirety on a hyper acid-labile resin, and mixing the highly pure Bivalirudin with at least one pharmaceutical acceptable excipient.

In yet another embodiment there is provided a method of treating a patient in need thereof comprising administering a therapeutically effective amount of a pharmaceutical composition comprising Bivalirudin having a purity of at least about 98.5% and at least one pharmaceutical acceptable excipient.

### DETAILED DESCRIPTION OF THE INVENTION

The invention encompasses methods for production of Bivalirudin of high purity. More specifically, the invention encompasses methods for the production of Bivalirudin in such a way that the peptide prepared and purified is a peptide of high purity. As used herein, the term “high purity” refers to a composition with a purity of at least about 98.5%. Furthermore, the term % purity as used herein relates to the % purity of the peptide in weight percent.

One of the advantages of the process of the present invention is that all synthetic steps are performed under mild conditions providing a low content of by-products and thereby a high yield and high purity of the final Bivalirudin peptide product. Another advantage is that it uses regular commercially available protected amino acids.

The peptides synthesized by one of the processes of the invention are prepared by using solid-phase synthesis using a hyper acid labile resin, extremely acid labile or super acid labile resin. Examples of the hyper acid-labile resins are well known in the art and are well described and referenced in Bodanszky et al., Principles of Peptide Synthesis, 2^{nd} ed., Springer Verlag Berlin Heidelberg 1989. Some examples are: 2-Cl -Trt-Cl resin®, a HMPB-BHA resin®, a Rink acid resin®, or a NovaSyn TGT alcohol resin®. The hyper acid-labile resins used in the method of the present invention allow cleavage of the synthesized peptide from the under mild acidic conditions, as the linkage of a peptide with such resin is susceptible to cleavage under mild acidic conditions. Accordingly, a suitable hyper acid-labile resin for preparing the Bivalirudin peptide according to the invention may be selected from the group consisting of a 2-Cl-Trt-Cl resin®, a HMPB-BHA resin®, a Rink acid resin®, or a NovaSyn TGT alcohol resin®. In a preferred embodiment, the hyper acid-labile resin used in the process of the invention is 2-Cl-Trt-Cl resin.

Due to the acid-lability of the solid phase attachment, such synthetic strategy employs Fmoc chemistry for carrying out the coupling reactions during solid phase synthesis, while only the terminating D-Phe residue may be either Boc or Fmoc protected. In a preferred embodiment of the present invention, Fmoc protection is used and may be eliminated from the peptide which remains on resin, by standard treatment with e.g. 20% piperidine or other Fmoc deprotecting base reagent known in the art to yield the peptide-resin conjugate. Such Fmoc deprotecting base reagents are, for example, a dilute solution of TFA in DCM, preferably 0.5% to 10% TFA in DCM (vol/vol), more preferably 1% to 5% TFA in DCM (vol/vol), even more preferably 1% to 2% TFA in DCM (vol/vol), most preferably 2% TFA in DCM (vol/vol), or a solution of acetic acid in DCM and Trifluoroethanol.

The first amino acid is attached to the resin via a highly acid labile ester linkage while other functional amino acid residues, other than the a-amine group, are protected by more stable protecting groups that are not cleaved or deprotected under the conditions required for the cleavage of the peptide from the resin. Such multi-functional amino acids are protected with a strong acid labile protecting group on the functional groups other than the α-amine group. These more acid stable protecting groups used on the other functional residues of the amino acids include, but are not limited to Pbf, tBu, Trt, and Boc, preferably Pbf for Arg residues and tBu, Trt and Boc for all other amino acid residues.

After completion of the synthesis of the Bivalirudin sequence, the protecting groups are removed using any conventional method. For example, one method includes, but is not limited to, a TFA based cocktail that contains in addition to TFA several scavengers such as EDT, DDM, phenol, thioanisole, and water. This uncoupling of a peptide or peptide fragments according to the present invention from the resin and deprotecting these peptides or peptide fragments of their protecting groups may be preformed in a one step process.

As used herein the term strong acidic solution refers to a solution of an acid which dissociates completely or almost completely. Weak and mild acids do not. Strong acids as used herein generally have a pKa less than about 1, preferably less than about 0.5.

The final peptide is purified by suitable methods to obtain a high purity peptide. Preferably, purification is carried out using reverse-phase HPLC (RP-HPLC).

For purposes of clarity and as an aid in understanding the invention, as disclosed and claimed herein, the following terms and abbreviations are defined below:

AA—Amino Acid

ACN—acetonitrile

Boc—t-Butyloxycarbonyl

BOP—Benzotriazole-1-yl-oxy-tris(dimethylamino)phosphonium hexafluorophosphate

Bzl—benzyl

Cbz—benzyloxycarbonyl

DBU—1,8-Diazobicyclo[5.4.0]undec-7-ene

DCM—dichloromethane

DCC—N,N′-Dicyclohexylcarbodiimide

DIC—1,3-Diisopropylcarbodiimide

DDM—dodecylmercaptane

DIPEA—diisopropylethylamine

DMF—dimethylformamide

EDT—ethanedithiol

Fmoc—9-fluorenylmethoxycarbonyl

HBTU—2-(1H-Benzotriazole-1-yl)-1,1,3,3-tetramethyluronium hexafluorophosphate

HOBt—N-hydroxybenzotriazole

MTBE—Methyltertiarybutylether

Pbf—pentamethyldihydrobenzofuransulfonyl

PyBOP—Benzotriazole-1-yl-oxy-tris-(pyrrolidino)-phosphonium hexafluorophosphate

SPPS—solid phase peptide synthesis

TBTU—O-Benzotriazole-1-yl-1,1,3,3-tetramethyluronium tetrafluoroborate

tBu—tert-Butyl ester

TFA—trifluoroacetic acid

TIS—triisopropylsilane

Trt—trityl

The term semi-protected peptide is used herein to describe a peptide which is unprotected with the exception of the presence of at least one but not all of the remaining protecting groups. Preferably, a semi-protected peptide is an unprotected peptide with the exception of the presence of a remaining α-amino N-protecting group.

In one embodiment of the present invention there is provided a method of preparing a high purity Bivalirudin comprising the following steps:

a) preparing a Bivalirudin peptide sequence on a hyper acid-labile resin, wherein the peptide contains suitably protected residues;

b) removing of the protected peptide from the resin using an acid solution containing at least one scavenger, to form an unprotected or semi-protected crude Bivalirudin peptide;

c) isolating the unprotected or semi-protected crude Bivalirudin peptide from the cleaving solution by precipitation or other suitable technique, and in case of a semi-protected crude Bivalirudin peptide removing any remaining protecting groups from the semi-protected crude Bivalirudin peptide to form an unprotected crude Bivalirudin peptide; and

d) purification of the crude Bivalirudin peptide by suitable method to obtain a Bivalirudin product.

Preferably, the obtained Bivalirudin product is dried to obtain a dry final Bivalirudin peptide of high purity. Preferably, drying the Bivalirudin product comprises lyophilization. Further, the resulting Bivalirudin peptide preferably has a purity of at least 98.5% purity, more preferably of at least 99.0% purity.

Preferably, isolating the crude peptide, preferably by for example precipitation, crystallization, extraction or chromatography, to produce an isolated crude peptide. Isolation of the unprotected or semi-protected crude Bivalirudin as in step (c) is preferably accomplished through precipitation of the peptide material. Precipitation of a crude peptide comprises using any solvent or mixtures of solvents which dissolve impurities and by products, while cause the precipitation of the peptide. Examples include, but are not limited to, a C₄ to C₈ alkyl ether, more preferably diethylether or MTBE, most preferably MTBE.

Preferably, purifying the crude Bivalirudin comprises purification by chromatography to obtain a peptide solution comprising a high purity Bivalirudin peptide and drying the peptide solution to obtain Bivalirudin of high purity. Preferably, drying of the peptide solution to obtain highly pure Bivalirudin is through lyophilization.

In another embodiment, the method for preparing high purity Bivalirudin comprises the following steps. In this method at least two fragments of the Bivalirudin peptide are prepared and are subsequently coupled to form Bivalirudin. The process comprises the steps of:

a) preparing a protected N-terminal fragment A of Bivalirudin on a hyper acid-labile resin and a protected fragment B of Bivalirudin on a hyper acid-labile resin, wherein the peptides contain suitably protected residues and at least the α-amino group of fragment B is protected by a Fmoc protecting group;

b) removing both peptides from their respective resins to form a protected fragment A and protected fragment B with a suitable cleaving solution;

c) coupling of the protected fragment B with Leu-OtBu to form an elongated fragment B;

d) deprotecting the α-amino protecting group Fmoc from the elongated fragment B by treatment with a suitable basic solution;

e) coupling protected fragment A with the elongated fragment B in solution by suitable method;

f) deprotecting all remaining acid labile protecting groups of the protected peptide by treatment with a suitable acidic solution containing at least one scavenger; and

g) purifying the crude Bivalirudin peptide by suitable method to form a Bivalirudin product of high purity, wherein Fragment A and Fragment B together form the peptide sequence D-Phe-Pro-Arg-Pro-Gly-Gly-Gly-Gly-Asn-Gly-Asp-Phe-Glu-Glu-Ile-Pro-Glu-Glu-Tyr-OH (SEQ ID No: 4).

Moreover, the fragments A and B after their removal from the hyper acid-labile resin, the Fmoc deprotected elongated fragment B and the crude Bivalirudin peptide are preferably isolated as fragments A and B, and crude Bivalirudin prior to their use in a subsequent step of the process of the invention. The optional isolation of fragments A and B, and crude Bivalirudin of the process of the invention preferably comprises precipitation in an ether, preferably a lower alkyl (C₄ to C₈) ether, more preferably MTBE.

Preferably, the strong acid solution for deprotecting the remaining protecting groups of the combined polypeptide of step (f) comprises a strong acid and at least one scavenger. Preferably, the purification of the crude Bivalirudin peptide comprises chromatography, preferably HPLC, and drying the peptide solution to obtain Bivalirudin of high purity, preferably through lyophilization.

This process for preparing Bivalirudin may further comprise purifying the semi-protected Bivalirudin peptide obtained after coupling step (e) before deprotecting step (f). This process for preparing Bivalirudin may further comprise purifying a semi-protected Bivalirudin peptide having any remaining α-amino protecting group and removing such remaining α-amino protecting group prior to purifying the crude Bivalirudin peptide as in step (g).

Preferably, in the above process, the hyper acid-labile resin used for preparing each of fragment A and fragment B is selected from the group consisting of a 2-Cl-Trt-Cl resin®, a HMPB-BHA resin®, a Rink acid resin®, and a NovaSyn TGT alcohol resin®. In a preferred embodiment the hyper acid-labile resin is 2-Cl-Trt-Cl resin.

The purity of the obtained Bivalirudin peptide prepared according to a process of the invention is at least 98.5% as measured by HPLC. Preferably, the purity of the obtained Bivalirudin peptide is at least 99% as measured by HPLC.

In the method of the present invention Fragment A and Fragment B together form the peptide sequence D-Phe-Pro-Arg-Pro-Gly-Gly-Gly-Gly-Asn-Gly-Asp-Phe-Glu-Glu-Ile-Pro-Glu-Glu-Tyr-OH (SEQ ID No: 4). Fragment A comprises the N-terminal sequence D-Phe-(AA)ₙ of the above amino acid sequence SEQ ID No:4, wherein n is an integer from 1-17, preferably from 3 to 15, more preferably from 5 to 12, most preferably from 8 to 10. Fragment B is a sequence comprising the remaining amino acids which complements fragment A to form a complete amino acid sequence of SEQ ID No:4, fragment B having a sequence of (AA)ₘ-Tyr-OH wherein m is an integer from 0-16, preferably from 2 to 14, more preferably from 5 to 12, most preferably from 7 to 9.

Suitable protecting groups for the terminal α-amine acid residue include, but are not limited to, 9-fluorenylmethoxycarbonyl (Fmoc) and BOC. A preferred terminal amino acid residue protecting group for fragment B is Fmoc. Other functional residues on the amino acids for use in the synthesis of Bivalirudin are protected with suitable protecting groups which include, but are not limited to, Pbf, tBu, Trt, and Boc, preferably Pbf for the Arg residues, and the tBu and Trt protecting groups for hydroxyl and carboxyl containing residues. A preferred protected Fragment A has the sequence [Xα-D-Phe-Pro-Arg(Pbf)-Pro-Gly-Gly-Gly-Gly-Asn(Trt)-Gly-OH] (SEQ ID No:8), wherein Xα represents a Boc or Fmoc protecting group. The preferred protected fragment B has the sequence [Fmoc-Asp(tBu)-Phe-Glu(tBu)-Glu(tBu)-Ile-Pro-Glu(tBu)-Glu(tBu)-Tyr(tBu)-OH] (SEQ ID No:9).

The peptide fragments A and B are removed from their respective hyper acid-labile resins using a suitable cleaving solution. Suitable cleaving solutions are mild acidic solutions comprising for example a dilute solution of trifluoroacetic acid (TFA) in DCM, or a solution of Acetic acid in DCM and Trifluoroethanol. A preferred mild acidic solution is a solution of TFA at a concentration of about 0.5 to about 10 vol/vol % in DCM, more preferably a solution of TFA at a concentration of about 1% to about 5 vol/vol % in DCM, even more preferably 1% to 2% TFA in DCM (vol/vol), most preferably 2% TFA in DCM (vol/vol), or a solution of acetic acid in DCM and Trifluoroethanol. The resulting acidic solution of the peptide may be neutralized immediately by equivalent amounts of a suitable base. A suitable base is any base which will neutralize the acidic solution, without removing a base-labile protecting group. Preferably, DIPEA or collidine may be used.

The preparation of a Bivalirudin peptide or a fragment thereof on a hyper acid-labile resin in the method of the present invention may be carried out by known methods of elongating a peptide chain on a solid resin. Preferably, the synthesis of the peptide sequence is carried out by a stepwise Fmoc SPPS (solid phase peptide synthesis) procedure which comprises the steps of loading a Fmoc protected first amino acid to a hyper acid-labile resin, preferably the resin is 2-Cl-Trt-Cl. Washing the resin and removing the Fmoc protecting group by treatment with a basic solution, preferably a solution of 20% piperidine in DMF. Washing to remove residual reagents and introducing the second Fmoc protected amino acid to start a first coupling step. The Fmoc protected amino acid is activated, preferably in situ, using a coupling agent, preferably TBTU/HOBt (N-hydroxybenzotriazole) and is subsequently coupled to the resin in the presence of an organic base, preferably Diisopropylethylamine. Washing the resin and removing the Fmoc protecting group on the α-amine by treatment with a basic solution, preferably a solution of 20% piperidine in DMF. These steps are repeated for each additional amino acid in the peptide sequence. Preferably, loading of the first Fmoc protected amino acid comprises stirring the hyper acid-labile resin with a solution of the Fmoc protected amino acid in an organic solvent, preferably DMF, in the presence of a coupling agent. Further, preferably three equivalents of the activated amino acids are employed in the coupling reactions.

The addition of amino acids to a peptide fragment or the coupling of peptide fragments A and B in the method of the present invention preferably uses coupling agents. Suitable coupling agents include, but are not limited to, 2-(1H-benzotriazole-1-yl)-1,1,3,3-tetramethyluronium tetrafluoroborate (TBTU), DCC, DIC, HBTU, BOP, or PyBOP. Coupling of a protected peptide with an amine containing compound is preferably carried out in a coupling solvent. Any solvent non-alcoholic solvents may be used as coupling solvents with the proviso that the solvent is inert in the coupling reaction. Preferably, the coupling solvent is selected from the group consisting of DMF, DMSO, DMA, NMP, DCM, and dioxane, more preferably the coupling solvent is DMF. This coupling solvent may also contain an organic base, preferably diisopropylethylamine (DIPEA) or Collidine. The carboxylic group of the protected peptide can be activated by a suitable method either in-situ or prior to the introduction of the amino compound in the reaction mixture.

Furthermore, in each step of the process of preparing Bivalirudin in which a chemical reaction is conducted, such as for example a coupling reaction, a washing step is preferably included for the removal of unreacted materials and other byproducts. Suitable solvents for use in the washing steps of the method of the present invention are dipolar solvents which do not interact with the peptide or resin. Water is not a preferred washing solvent as it causes partial hydrolysis of the peptide and interacts with the resin. Preferred solvents for a washing step include, but are not limited to, dimethylformamide (DMF), dichloromethane (DCM), methanol (MeOH), or isopropanol (IPA).

The terminal amino acid residue Fmoc protecting group is removed by any known method using suitable basic solutions, such as a reaction with a piperidine solution in DMF. Other basic suitable solutions include, but are not limited to, solutions of DBU, DBU/piperidine, and diethylamine in an inert solvent.

Deprotection of the acid-labile protecting groups from the peptide may be effected by addition of a strong acidic solution. The strong acidic solution preferably comprises an acid, such as TFA, TFMSA, HBr/AcOH, and HF, at least one scavenger reagent including, but not limited to, ethanedithiol (EDT), thioanisole, TIS, DDM, phenol, and m-cresol, and water. The relative ratio of acidic material to scavenger to water in the strong acid solution used in the present invention preferably comprises from about 85% to about 99% acid, from about 0.1% to about 15% scavenger, and from about 0.1% to about 15% water by weight. A preferred strong acidic solution comprises about 95% TFA, about 2.5% EDT, and about 2.5% water by weight.

The crude Bivalirudin peptide product may be purified by any known method. Preferably, the peptide is purified using HPLC on a reverse phase (RP) column. A preferred method of purifying the crude Bivalirudin peptide comprises a HPLC system with a reverse phase C₁₈ column. The resulting purified product is preferably dried, more preferably lyophilized. The obtained highly purified Bivalirudin has a purity of at least about 98.5% as measured by HPLC, wherein the total impurities amount to less than 1.5% as measured by HPLC, comprising not more than 0.5% as measured by HPLC [Asp⁹-Bivalirudin] and each is impurity less than 1.0% as measured by HPLC. Preferably, the highly purified Bivalirudin has a purity of at least about 99.0% as measured by HPLC, wherein the total impurities amount to less than 1.0% as measured by HPLC, comprising not more than 0.5% [Asp⁹-Bivalirudin] as measured by HPLC and each impurity is preferably less than 0.5% as measured by HPLC. A suitable method for the determination of the purity of the Bivalirudin peptide includes, but is not limited to, using HPLC. A preferred method of determining the purity of the Bivalirudin peptide comprises a HPLC system with a reverse phase C₁₂ column, wherein the peptide is eluted with a gradient of TFA in water/acetonitrile.

In another embodiment there is provided a pharmaceutical composition comprising highly pure Bivalirudin having a purity of at least about 98.5% as measured by HPLC and at least one pharmaceutical acceptable excipient.

Further, in another embodiment there is provided a method of preparing a pharmaceutical composition comprising Bivalirudin having a purity of at least 98.5% as measured by HPLC, comprising preparing highly pure Bivalirudin, either in fragments or in its entirety on a hyper acid-labile resin, and mixing the highly pure Bivalirudin with at least one pharmaceutical acceptable excipient.

Pharmaceutical formulations of the present invention contain highly purified Bivalirudin. The highly purified Bivalirudin prepared by the processes of the present invention are ideal for pharmaceutical formulation. In addition to the active ingredient(s), the pharmaceutical compositions of the present invention may contain one or more excipients. Excipients are added to the composition for a variety of purposes.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac Di Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, highly purified Bivalirudin and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), and inhalant administration. Although the most suitable administration in any given case will depend on the nature and severity of the condition being treated, the most preferred route of the present invention is parenteral. The dosages may be conveniently presented in unit dosage form and prepared by any of the methods well known in the pharmaceutical arts.

Dosage forms include solid dosage forms like tablets, powders, preferably lyophilized powder compositions, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art. The dosage of pharmaceutically acceptable compositions described in U.S. Pat. No. 5,196,404 may be used as a guidance.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The dosage is preferably in the form of an infusion solution administered as an intravenous bolus dose or by infusion. When administered as an intravenous bolus dose the preferred dose is about 0.75 mg/kg. The preferred infusion dose is about 1.75 mg/kg/h.

In another embodiment there is provided a method of treating a patient in need thereof comprising administering a therapeutically effective amount of a pharmaceutical composition comprising Bivalirudin having a purity of at least about 98.5% as measured by HPLC, and at least one pharmaceutical acceptable excipient. Preferably, the method is to administer an anticoagulant in patients with unstable angina undergoing percutaneous transluminal coronary angioplasty (PTCA) or in patients undergoing percutaneous coronary intervention.

Having described the invention with reference to certain preferred embodiments, other embodiments will become apparent to one skilled in the art from consideration of the specification. The disclosures of the prior art references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Example 1

Preparation of High Purity Bivalirudin by Sequential Solid Phase Synthesis

Synthesis of the peptide sequence was carried out by a stepwise Fmoc SPPS (solid phase peptide synthesis) procedure starting with loading a Fmoc-Leu-OH to 2-Cl-Trt-Cl resin. The resin (2-Cl-Trt-Cl resin, 20 g) after washing was stirred with a solution of Fmoc-Leu-OH (17.0 g) in DMF in the presence of diisopropylethylamine for 2 h. After washing of the resin the Fmoc protecting group was removed by treatment with 20% piperidine in DMF. After washing of residual reagents the second amino acid (Fmoc-Tyr(tBu)) was introduced to start the first coupling step. The Fmoc protected amino acid was activated in situ using TBTU/HOBt (N-hydroxybenzotriazole) and subsequently coupled to the resin for 50 minutes. Diisopropylethylamine was used during coupling as an organic base. Completion of the coupling was indicated by a Ninhydrine test. After washing of the resin, the Fmoc protecting group on the α-amine was removed with 20% piperidine in DMF for 20 min. These steps were repeated each time with another amino acid according to peptide sequence. All amino acids used were Fmoc-N^{α} protected except the last amino acid in the sequence, Boc-D-Phe. Trifunctional amino acids were side chain protected as follows: Ser(tBu), Arg(Pbf), Tyr(tBu), Asp(OtBu) and Glu(OtBu). Three equivalents of the activated amino acids were employed in the coupling reactions. At the end of the synthesis the peptide-resin was washed with DMF, followed by MeOH, and dried under vacuum to obtain 57 g dry peptide-resin.

The cleavage of the peptide from the resin with simultaneous deprotection of the protecting groups was performed as following: a. 57 g peptide resin obtained as described above were added to the reactor containing a cold solution of 95% TFA, 2.5% TIS, 2.5% EDT; b the mixture was mixed for 2 hours at room temperature; c. the product was precipitated by the addition of 10 volumes of ether (MTBE), filtered and dried in vacuum to obtain 31.7 g crude product.

The crude peptide (31.7 g) obtained above, was dissolved in aqueous solution of acetonitrile. The resulting solution was loaded on a C₁₈ RP-HPLC column and purified to obtain fractions containing Bivalirudin at a purity of >97.5%. The pure fractions were collected and lyophilized to obtain a final dry peptide (4.4 g) which is at least 99.0% pure (HPLC). It contained not more than 0.5% [Asp⁹-Bivalirudin] and not more than 0.5% of any impurity. The purity of the Bivalirudin was determined with HPLC on a Synergi C₁₂ Max-RP (250×4.6 mm, 4 μm) column. The mobile phase A was 0.05% (v/v) TFA in water and the mobile phase B 0.05% (v/v) TFA in acetonitrile. The following gradient was applied to the column loaded with 25 μl of sample, at t₀: A=83%, B=17%, at t₃₀ A=60%, B=40%, at t₃₃ A=10%, B=90%, and at t₃₈ A=10%, B=90%. The flow rate was 1.0 ml/min at an oven temperature of 40° C. The UV-detector was set at 214 nm.

### Example 2

Preparation of Protected Fragment A [Boc-D-Phe-Pro-Arg(Pbf)-Pro-Gly-Gly-Gly-Gly-Asn(Trt)-Gly-OH] (SEQ ID No:10)

Synthesis of the protected peptide was carried out by a stepwise Fmoc SPPS (solid phase peptide synthesis) procedure starting with loading a Fmoc-Gly-OH to 2-Cl-Trt-Cl resin. The resin (2-Cl-Trt-Cl resin, 500 g) after washing was stirred with a solution of Fmoc-Gly-OH in DMF in the presence of diisopropylethylamine for 2 h. After washing of the resin the Fmoc protecting group was removed by treatment with 20% piperidine in DMF. After washing of residual reagents the second amino acid (Fmoc-Asn(Trt)-OH) was introduced to start the first coupling step. The Fmoc protected amino acid was activated in situ using TBTU/HOBt (N-hydroxybenzotriazole) and subsequently coupled to the resin for 50 minutes. Diisopropylethylamine or Collidine were used during coupling as an organic base. Completion of the coupling was indicated by a Ninhydrine test. After washing of the resin, the Fmoc protecting group on the α-amine was removed with 20% piperidine in DMF for 20 min. These steps were repeated each time with another amino acid according to peptide sequence. All amino acids used were Fmoc-N⁶⁰ protected except the last amino acid in the sequence, Boc-Phe-OH. Trifunctional amino acids were side chain protected as follows: Arg(Pbf)-OH and Asn(Trt)-OH. Three equivalents of the activated amino acids were employed in the coupling reactions. At the end of the synthesis the peptide-resin was washed with DMF, followed by DCM, and dried under vacuum to obtain 1200 g of dry peptide-resin.

The peptide, prepared as described above, was cleaved from the resin using a 1% TFA solution in DCM by three repeated washings (15 min each). The acidic peptide solution was neutralized by DIPEA. The solvent was evaporated under reduced pressure and the protected peptide was precipitated by the addition of 10 volumes of water, filtered and dried in vacuum to obtain 680 g powder. It was identified as Boc-D-Phe-Pro-Arg(Pbf)-Pro-Gly-Gly-Gly-Gly-Asn(Trt)-Gly-OH (SEQ ID No:10).

### Example 3

Preparation of Protected Fragment B [Fmoc-Asp(tBu)-Phe-Glu(tBu)-Glu(tBu)-Ile-Pro-Glu(tBu)-Glu(tBu)-Tyr(tBu)-OH] (SEQ ID No:9)

Synthesis of the protected peptide was carried out by a stepwise Fmoc SPPS (solid phase peptide synthesis) procedure starting with loading a Fmoc-Tyr(tBu)-OH to 2-Cl-Trt-Cl resin. The resin (2-Cl-Trt-Cl resin, 1000 g) after washing was stirred with a solution of Fmoc-Tyr(tBu)-OH in DMF in the presence of diisopropylethylamine for 2 h. After washing of the resin the Fmoc protecting group was removed by treatment with 20% piperidine in DMF. After washing of residual reagents the second amino acid (Fmoc-Glu(OtBu)-OH) was introduced to start the first coupling step. The Fmoc protected amino acid was activated in situ using TBTU/HOBt (N-hydroxybenzotriazole) and subsequently coupled to the resin for 50 minutes. Diisopropylethylamine or Collidine were used during coupling as an organic base. Completion of the coupling was indicated by a Ninhydrine test. After washing of the resin, the Fmoc protecting group on the α-amine was removed with 20% piperidine in DMF for 20 min. These steps were repeated each time with another amino acid according to peptide sequence. All amino acids used were Fmoc-N⁶⁰ protected. Trifunctional amino acids were side chain protected as follows: Glu(OtBu)-OH and Asp(OtBu)-OH. Three equivalents of the activated amino acids were employed in the coupling reactions. At the end of the synthesis the peptide-resin was washed with DMF, followed by DCM, and dried under vacuum to obtain 2600 g of dry peptide-resin.

The peptide, prepared as described above, was cleaved from the resin using a 1% TFA solution in DCM by three repeated washings (15 min each). The acidic peptide solution was neutralized by DIPEA. The solvent was evaporated under reduced pressure and the protected peptide was precipitated by the addition of 10 volumes of water, filtered and dried in vacuum to obtain 1650 g powder. It was identified as Fmoc-Asp(tBu)-Phe-Glu(tBu)-Glu(tBu)-Ile-Pro-Glu(tBu)-Glu(tBu)-Tyr(tBu)-OH (SEQ ID No:9).

### Example 4

Preparation of Asp(tBu)-Phe-Glu(tBu)-Glu(tBu)-Ile-Pro-Glu(tBu)-Glu(tBu)-Tyr(tBu)-Leu-OtBu (SEQ ID No:11)

Fmoc-Asp(tBu)-Phe-Glu(tBu)-Glu(tBu)-Ile-Pro-Glu(tBu)-Glu(tBu)-Tyr(tBu)-OH (SEQ ID No:9) (1650 g) was dissolved in DMF and Leu-OtBu (224 g) was added at room temperature. The mixture was agitated in the reactor and cooled to −5 ° C. A solution of HOBt in DMF (153 g in 300 ml) was added followed by a solution of TBTU in DMF (321 g in 1 L). Finally DIPEA (340 ml) was added and the reaction was continued for 3 h at room temperature. Completion of the reaction was monitored by HPLC analysis.

The Fmoc group was removed by addition of Piperidine (450 ml) into the reaction mixture at room temperature. The completion of the reaction was monitored by HPLC. The mixture was concentrated by partial evaporation of DMF under reduced pressure. The protected peptide was precipitated by addition of water. It was separated, washed and dried to obtain 1575 g of powder. It was identified as Asp(tBu)-Phe-Glu(tBu)-Glu(tBu)-Ile-Pro-Glu(tBu)-Glu(tBu)-Tyr(tBu)-Leu-OtBu (SEQ ID No:11).

### Example 5

Preparation of Bivalirudin

Boc-D-Phe-Pro-Arg(Pbf)-Pro-Gly-Gly-Gly-Gly-Asn(Trt)-Gly-OH (SEQ ID No:10) (170 g) and Asp(tBu)-Phe-Glu(tBu)-Glu(tBu)-Ile-Pro-Glu(tBu)-Glu(tBu)-Tyr(tBu)-Leu-OtBu (SEQ ID No:11) (252 g) were dissolved in DMF (2 L). Collidine (20 ml) was added followed by addition of TBTU solution in DMF (35 g in 180 ml). The mixture was stirred at room temperature and another portion of TBTU and Collidine were added after 2 h to bring the reaction to completion. On completion of the coupling reaction (monitored by HPLC) DMF was evaporated under reduced pressure and the protected Bivalirudin was precipitated in water. The precipitate was dried to obtain 416 g Boc-D-Phe-Pro-Arg(Pbf)-Pro-Gly-Gly-Gly-Gly-Asn(Trt)-Gly-Asp(tBu)-Phe-Glu(tBu)-Glu(tBu)-Ile-Pro-Glu(tBu)-Glu(tBu)-Tyr(tBu)-Leu-OtBu (SEQ ID No:12).

The protected Bivalirudin was dissolved in a cold TFA solution containing 5% DDM and 2.5% water. The solution was stirred at room temperature for 1 h. It was concentrated on a rotavapor and added to cold MTBE (10 volumes). Precipitated Bivalirudin was separated by filtration and dried to obtain 355 g crude product.

The crude peptide (355 g) obtained above, was dissolved in an aqueous solution of acetonitrile. The resulting solution was loaded on a C₁₈ RP-HPLC column and purified to obtain fractions containing Bivalirudin at a purity of >97.5%. The pure fractions were collected and lyophilized to obtain a final dry peptide (110 g) which is at least 99.0% pure (HPLC). It contained not more than 0.5% [Asp⁹-Bivalirudin] and not more than 0.5% of any impurity.

1-49. (canceled)

50. A composition of hybrid synthetic Bivalirudin having a purity of at least about 98.5% as measured by high performance liquid chromatography (HPLC) after purification.

51. The composition of claim 50, wherein the hybrid synthetic Bivalirudin has Asp⁹-Bivalirudin in an amount no greater than 0.5% as measured by HPLC.

52. The composition of claim 50, wherein the hybrid synthetic Bivalirudin comprises one or more impurities, wherein each impurity is less than 1.0% as measured by HPLC.

53. The composition of claim 50, wherein the hybrid synthetic Bivalirudin has a purity of at least about 99.0% as measured by HPLC.

54. The composition of claim 53, wherein the hybrid synthetic Bivalirudin comprises one or more impurities, wherein each impurity is less than 0.5% as measured by HPLC.

55. A composition of hybrid synthetic Bivalirudin comprising one or more impurities, wherein each impurity is less than 1.0% as measured by high performance liquid chromatography (HPLC) after purification.

56. The composition of claim 55, wherein the hybrid synthetic Bivalirudin has Asp⁹-Bivalirudin in an amount no greater than 0.5% as measured by HPLC.

57. The composition of claim 55, wherein the hybrid synthetic Bivalirudin has a purity of at least about 99.0% as measured by HPLC.

58. The composition of claim 57, wherein each impurity is less than 0.5% as measured by HPLC.

59. A pharmaceutical composition comprising hybrid synthetic Bivalirudin and at least one pharmaceutical acceptable excipient, wherein the Bivalirudin has a purity of at least about 98.5% as measured by high performance liquid chromatography (HPLC) after purification.

60. The pharmaceutical composition of claim 59, wherein the at least one pharmaceutical acceptable excipient comprises mannitol.

61. The pharmaceutical composition of claim 59, wherein the pharmaceutical composition is in a solid dosage form.

62. The pharmaceutical composition of claim 61, wherein the solid dosage form is a powder.

63. The pharmaceutical composition of claim 59, wherein the pharmaceutical composition is in the form of an infusion solution.

64. The pharmaceutical composition of claim 59, wherein the hybrid synthetic Bivalirudin has Asp⁹-Bivalirudin in an amount no greater than 0.5% as measured by HPLC.

65. The pharmaceutical composition of claim 59, wherein the hybrid synthetic Bivalirudin comprises one or more impurities, wherein each impurity is less than 1.0% as measured by HPLC.

66. The pharmaceutical composition of claim 59, wherein the hybrid synthetic Bivalirudin has a purity of at least about 99.0% as measured by HPLC.

67. The pharmaceutical composition of claim 66, wherein the hybrid synthetic Bivalirudin comprises one or more impurities, wherein each impurity is less than 0.5% as measured by HPLC.

68. A pharmaceutical composition comprising hybrid synthetic Bivalirudin and at least one pharmaceutical acceptable excipient, wherein the Bivalirudin comprises one or more impurities, wherein each impurity is less than 1.0% as measured by high performance liquid chromatography (HPLC) after purification.

69. The pharmaceutical composition of claim 68, wherein the at least one pharmaceutical acceptable excipient comprises mannitol.

70. The pharmaceutical composition of claim 68, wherein the pharmaceutical composition is in a solid dosage form.

71. The pharmaceutical composition of claim 70, wherein the solid dosage form is a powder.

72. The pharmaceutical composition of claim 68, wherein the pharmaceutical composition is in the form of an infusion solution.

73. The pharmaceutical composition of claim 68, wherein the hybrid synthetic Bivalirudin has Asp⁹-Bivalirudin in an amount no greater than 0.5% as measured by HPLC.

74. The pharmaceutical composition of claim 68, wherein the hybrid synthetic Bivalirudin has a purity of at least about 99.0% as measured by HPLC.

75. The pharmaceutical composition of claim 74, wherein each impurity is less than 0.5% as measured by HPLC.


SEQUENCE LISTING (12 sequences)

SEQ ID NO: 1 - protein, 20 aa, Artificial Sequence
  Description of Artificial Sequence Synthetic peptide
FPRPGGGGNGDFEEIPEEYL

SEQ ID NO: 2 - protein, 10 aa, Artificial Sequence
  Description of Artificial Sequence Synthetic peptide
FPRPGGGGNG

SEQ ID NO: 3 - protein, 9 aa, Artificial Sequence
  Description of Artificial Sequence Synthetic peptide
DFEEIPEEY

SEQ ID NO: 4 - protein, 19 aa, Artificial Sequence
  Description of Artificial Sequence Synthetic peptide
FPRPGGGGNGDFEEIPEEY

SEQ ID NO: 5 - protein, 10 aa, Artificial Sequence
  Description of Artificial Sequence Synthetic peptide
DFEEIPEEYL

SEQ ID NO: 6 - protein, 20 aa, Artificial Sequence
  Description of Artificial Sequence Synthetic peptide
FPRPGGGGNGDFEEIPEEYL

SEQ ID NO: 7 - protein, 20 aa, Artificial Sequence
  Description of Artificial Sequence Synthetic peptide
FPRPGGGGNGDFEEIPEEYL

SEQ ID NO: 8 - protein, 10 aa, Artificial Sequence
  Description of Artificial Sequence Synthetic peptide
FPRPGGGGNG

SEQ ID NO: 9 - protein, 9 aa, Artificial Sequence
  Description of Artificial Sequence Synthetic peptide
DFEEIPEEY

SEQ ID NO: 10 - protein, 10 aa, Artificial Sequence
  Description of Artificial Sequence Synthetic peptide
FPRPGGGGNG

SEQ ID NO: 11 - protein, 10 aa, Artificial Sequence
  Description of Artificial Sequence Synthetic peptide
DFEEIPEEYL

SEQ ID NO: 12 - protein, 20 aa, Artificial Sequence
  Description of Artificial Sequence Synthetic peptide
FPRPGGGGNGDFEEIPEEYL